(19) United States
(12) Patent Application Publication
(10) Pub. No.: US 2012/0045543 A1
(43) Pub. Date: Feb. 23, 2012

(54) SHELF-LIFE EXTENSION OF SOUR DOUGH BAKED GOODS

(75) Inventors: Markus Brandt, Minden (DE); Judith Amann-Brandt, Port-Westfalica (DE)

(73) Assignee: ERNST BÖCKER GMBH & CO. KG, Minden (DE)

(21) Appl. No.: 13/266,028

(22) PCT Filed: Apr. 23, 2010
(86) PCT No.: PCT/EP2010/055489

§ 371 Date: Oct. 24, 2011

(30) Foreign Application Priority Data
Apr. 24, 2009 (DE) 09158710.5

(51) Int. Cl. A21D 8/04; C12N 1/21; A21D 13/00; C12N 1/20

(52) U.S. Cl. 426/18; 435/252.3; 435/252.9; 426/61; 426/549

(57) ABSTRACT

The present invention relates to an improved method for producing sour dough and baked goods having an extended shelf life, wherein biological preservatives are augmented in the dough or the baked goods by cofermenting selected lactobacilli.

The present invention relates to an improved method for the production of sour dough and baked goods having an extended shelf life, wherein, by co-fermentation of selected lactobacilli, biological preservatives are enriched in the dough or the baked goods.

The production of ready-to-eat foods using fermented cereals belongs to the oldest biotechnological processes employed by humans. The highest proportions in terms of quantity are accounted for by the production of bread. Thus, the per capita consumption of bread in the cereal economic year 2002/2003 was 86.6 kg.

At least 20% of the bread in Germany is produced using sour dough or dough acidification agents. An addition of sour dough causes, inter alia, owing to the reduction of the pH, an extended shelf life of bread. Nevertheless, some valuable foods such as, for example, wheat bread, are lost to human nutrition owing to spoilage. Since bread is increasingly frequently presented on the market in sliced form, for example toast bread, owing to the increased surface area, this has a higher risk of contamination, e.g. by molds. The main representatives in this case include the genera Penicillium sp., Aspergillus sp., Rhizopus sp., Mucor sp., but also yeast types of what is termed “chalk mold”.

The consumption of such contaminated products is a health hazard for the end consumer, since mold fungi can form mycotoxins. Therefore, the control of mold in bread is necessary both for reasons of health and also economics.

Measures for extending the shelf life of bread and baked goods are primarily directed towards delaying mold formation. The principles of the methods are divided into chemical methods such as, e.g. the addition of permitted preservatives, physical methods, such as thermal product treatment and biological methods.

The biological methods comprise here the use of sour dough and sour dough starter cultures, since the metabolic activities of the microorganisms participating in the sour dough fermentation permit “baking-active” components to be generated biologically, which components usually need to be added to the bread dough in the form of ingredients that must be declared. Examples thereof are, inter alia, the formation of exopolysaccharides by lactobacilli, which delay the staling of bread, or else the formation of antimicrobially acting acetic acid in the bread dough. Unfortunately, however, the formation of acetic acid is greatly restricted, in particular in wheat sour doughs.

However there is great economic interest in offering declaration-free products extending the shelf life.

It was therefore the purpose of this work to provide and optionally optimize starter cultures that have a delaying action on bread spoilage. The present invention, by using microorganisms as starter cultures, which microorganisms normally rarely participate in sour dough fermentation, opens up new ways for further optimizing bread quality.

It is due to the achievements and findings of the inventors that microorganisms which are not customarily concerned with bread baking and which are able to synthesize propionic acid were able to be found and used for enriching sour dough starter cultures and therefore for bread baking.

The invention accordingly teaches the use and employment of microorganisms in sour dough which, in addition to the lactic acid fermentation, also have the property of forming other organic acids. In particular, the invention relates to lactic acid bacteria which, in the sour dough, are able to form acetic acid, in amounts markedly above the usual level, and/or propionic acid.

Using an appropriate starter culture, during the dough production, significant amounts of acetic acid and/or propionic acid could be formed in the dough directly which then enhance the shelf life of the baked goods and delay the spoilage thereof by, e.g., fungal infestation. A problem in this context is providing starter cultures which have a desirable equilibrium of metabolic products since if e.g. too much propionic acid is formed, the baked product has a cheese-like unappetizing odor.

It is known that acetic acid and soluble salts thereof exhibit a slightly antimicrobial effect. This results especially from their pH-lowering action within the microorganism cell. Owing to the lipid nature of the microbial cytoplasmic membrane, only the undissociated fat-soluble acid can penetrate into the cell. The proportion of undissociated acetic acid is higher the lower is the pH in the food. In rye sour doughs, up to 0.4% acetic acid may form in the sour dough. At a sufficiently high usage rate of sour dough, this is sufficient to protect against mold fungi. In contrast, in the substrate wheat, only 0.03% is formed. The addition of acetic acid as a preservative to wheat doughs is not suitable since flavor changes and impairments occur. Therefore, frequently, for preserving wheat breads and for protection against spoilage by rope-producing bacteria (Bacillus spec.) and mold fungi, sodium diacetate is added as a source of acetic acid.

In addition, it is known that propionic acid and the readily soluble salts thereof show an antimicrobial activity. Propionic acid acts in this case by enzyme inhibition by accumulating in the cell and blocking the metabolism. In addition, propionic acid competes with other necessary growth substances, especially alanine and other amino acids. In the case of oral intake, propionic acid and propionates are not acutely toxic and do not have any recognizable toxic potential. They are readily absorbed by the digestive tract and are either utilized calorically in the body or incorporated into carbohydrates and fats. Since the end of the 1930s, propionates have been added in the USA for preserving bread and cheese.

The specific use of propionic acid and salts thereof, predominantly in low-acid white bread, proceeded for the first time in 1938, after in 1906 the activity of organic acids against rope formation in baked goods had already been known. Depending on the fungal species, the growth of mold fungi is inhibited at a pH of 5.5 by concentrations of 0.0125-1.25% propionic acid. For the inhibition of bacteria, higher concentrations of 1.6-6% are necessary.

Propionic acid is formed by various microorganisms, e.g. by Propionibacterium. In this case propionic acid bacteria use lactate as the main starting product in order to form, from 3 mol of lactate−>2 mol of propionate+1 mol of acetate+1 mol of CO₂. Propionic acid bacteria are Gram positive, customarily have the enzyme catalase and are facultatively anaerobic to aerotolerant. The pH optimum for multiplication is between 6 and 7.

Unfortunately it has been found that below pH 5 the growth rate is drastically reduced and only very little propionic acid is formed, as a result of which addition of propionic acid bacteria is hardly logical for the production of sour doughs.

Nevertheless the usage of propionic acid bacteria for establishing a protecting culture and thereby for inhibiting mold fungi in milk products or wheat breads has been described (Suomalainen & Mäyrä-Mäkinen, 1999, Fermented Breads and Milk 79, 165-174). In this case also, however, it has proved disadvantageous that propionic acid bacteria grow somewhat poorly at low pHs and form too little propionic acid.

The methods known in the prior art for the biological enrichment of doughs are therefore very limited, and sour doughs which customarily have a pH of <4 have not been able to be enriched to date by such biological methods with relatively high amounts of acetic acid and propionic acid.

There is therefore a need to provide novel biological methods in order to achieve an enrichment of organic acids for preservation purposes in bread and baked goods made of sour dough. In particular, it is therefore the object of the invention to provide biological methods which make possible an enrichment of sour doughs and baked goods with relatively high amounts of acetic acid and/or propionic acid.

For this purpose the invention provides a novel starter culture mixture for sour doughs which consists of strains that are selected from the group of the lactic acid bacteria, wherein at least one lactobacillus strain is selected which is able to form propionic acid.

Preferably, the strains are selected such that they can utilize a lactate-catabolizing metabolic pathway for the production of propionic acid that is different from the known propionic acid bacteria. According to one embodiment, the lactobacilli added to the starter culture metabolize, e.g. lactic acid, to form 1,2-propanediol or propanediol and form propionic acid therefrom.

Typically, the lactic acid bacteria added to the sour dough starter culture are selected from the group consisting of Lb. diolivorans, Lb. buchneri, Lb. parabuchneri, L. brevis, L. reuteri, Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confusa, P. acidilactici, P. parvulus and P. pentosaceus. According to a further embodiment, the lactic acid bacteria added to the starter culture are pure cultures.

Preferably, at least one strain of the lactic acid bacteria added to the sour dough starter culture is selected from the group Lb. diolivorans and lactobacilli or recombinant lactobacilli which are functionally able to form propionic acid, and preferably to convert propanediol to propionic acid. In sour doughs which are made up and fermented with such a starter culture, a significant enrichment of organic acids, and in particular propionic acid, is detectable.

According to one embodiment, therefore the sour dough starter culture, and also the finished sour dough, or a sour dough concentrate obtained therefrom, contains the microorganism Lactobacillus diolivorans. By means of a molecular-biological analysis, the microorganism Lactobacillus diolivorans can also be detected from the finished baked goods.

Further preferably, at least one second strain of the lactic acid bacteria added to the sour dough starter culture is selected from the group consisting of Lb. buchneri, Lb. parabuchneri, L. brevis, L. bifermentans and lactobacilli or recombinant lactobacilli which are functionally able to catabolize lactic acid to acetic acid and/or succinic acid. In sour doughs which are made up and fermented with such a starter culture, a significant enrichment of organic acids and acetic acid is detectable.

According to one embodiment, therefore, the sour dough starter culture and also the finished sour dough or a sour dough concentrate obtained therefrom, contains the microorganism Lactobacillus buchneri or Lb. parabuchneri. By means of a molecular-biological analysis, the microorganism Lactobacillus buchneri, L. bifermentans, L. reuteri, or Lb. parabuchneri can also be detected from the finished baked goods.

In the present application, the expression “lactobacilli or recombinant lactobacilli which are functionally able to form propionic acid” includes naturally discoverable and genetically engineered lactobacilli which, owing to their range of enzymes, are able to form propanediol, propionic acid or succinic acid. In particular, those lactobacilli are included which are able to convert propanediol to propionic acid.

To the method for producing sour dough, or to the sour dough starter culture relevant to the invention, the lactic acid bacteria strains are added which are selected from the group consisting of Lb. diolivorans, Lb. buchneri, Lb. parabuchneri, Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confuse, P. acidilactici, P. parvulus and P. pentosaceus, L. reutrei, L. brevis. The lactobacilli selected from this group for the starter dough or the starter culture serve for the usual sour dough production by lactic acid fermentation and produce in part metabolic intermediates which can be used for further reaction to form propionic acid or for increased acetic acid formation.

According to one embodiment, to the method for producing sour dough, or to the sour dough starter culture relevant to the invention, there are added at least the lactic acid bacteria strains selected from the group consisting of Lb. diolivorans, Lb. buchneri, Lb. parabuchneri—as indicator organisms. The addition of further strains selected from the group consisting of Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confuse, P. acidilactici, P. parvulus and P. pentosaceus is optional.

Using the production method according to the invention, according to further embodiments, in a period of approximately 8-100 days, preferably 10, 12, 14, 16 or 30 days, a sour dough or sour preliminary dough and, further, baked goods, can be produced which contain between 0.1% and 0.9% w/w, preferably 0.2%-0.4% w/w, preferably 0.3%-0.6% w/w, preferably 0.4%-0.8% w/w, propionic acid, relative to the initial flour weight. Further preferably, the sour dough, or a sour dough concentrate produced from this sour dough by reduction, produced by the method according to the invention contains about 0.6%-0.9% w/w, 0.8%-1.5% w/w, 1.0%-2.5% w/w, 1.9%-3.5% w/w, 2.0%-4.5% w/w, 3.0%-5.0% w/w, 4.0%-6.5% w/w, propionic acid, based on the initial flour weight.

The acetic acid content can be set using the method according to the invention, according to one embodiment, in a fermentation time period of approximately 8-100 days, preferably 10, 12, 14, 16 or 30 days, to a content of at least 0.6% w/w and up to about 6% w/w of acetic acid, based on the initial flour weight. Preferably, the sour dough or a sour dough concentrate produced from this sour dough by reduction, produced by the method according to the invention contains about 0.6%-0.9% w/w, 0.8%-1.2% w/w, 0.9%-1.8% w/w, 1.0%-2.0% w/w, 2.0%-6.0% w/w, of acetic acid, based on the initial flour weight.

The isolated or conjoint use of the strains Lb. diolivorans, Lb. buchneri, Lb. parabuchneri in starter cultures according to the invention for producing sour dough and, further, for producing sour dough concentrate, offers the further advantage that a correspondingly produced sour dough concentrate is stable over a plurality of months, and readily up to half a year. Stability in this context is taken to mean that the cell count (viable cell count) of the lactic acid bacteria remains constant over the stated time period. In conventional sour doughs or concentrates, the growth conditions change over the course of time greatly to the disadvantage of the microbes present, via the increase in lactic acid content. In consequence, the lactic acid bacteria die owing to the excessively acidic environment. Such a sour dough or concentrate can no longer be used as a baking raising agent.

In the sour dough or concentrate according to the invention, the strains used, in particular the strains Lb. diolivorans, Lb. buchneri or Lb. parabuchneri, catabolize lactic acid to form other organic acids. The growth conditions therefore remain acceptable for all other lactic acid bacteria present and they do not die off—even after a plurality of months.

As indicator strain, therefore, according to one embodiment of the composition of the starter culture or sour dough according to the present invention, Lb. diolivorans can be used. Preferably, the composition of the starter culture or of the sour dough according to the present invention contains at least 10⁶-10⁸ lactobacilli per liter, wherein approximately 10-50% are Lb. diolivorans.

By means of the production method according to the invention, according to further embodiments for preserving pasty and liquid sour dough products and baked goods resulting therefrom can be usable.

It is particularly noteworthy that, although the production method according to the invention does not lead to very high propionic acid amounts in the dough and later in the baked goods, by means of the combination of the acid formed by lactobacilli fermentation, that is to say lactic acid, acetic acid and propionic acid, an improved preservation can be achieved with relatively low propionic acid amounts.

Using the method known to those skilled in the art, the loss of propionic acid was determined in order to find out how much propionic acid is present in the various intermediates (dough versus baked product). It was found that, during baking and up to 24 h thereafter, the concentration of propionic acid remained roughly constant. This may be explained by the fact that at the end of the baking time the bread crumb has a temperature of at most 98° C.-106° C. Propionic acid has a boiling point of 141° C. and is therefore retained in the bread crumb during the baking to completion and also after cooling, and therefore can effect the desired protection against mold.

In the storage studies carried out, the inventors have found that in a reference experiment between propionic acid-free bread and bread to which propionic acid had been added, the bread to which 0.4% propionic acid was added exhibited a mold-free time that was 14 days longer compared with the propionic acid-free bread.

On inoculated bread slices, the growth comparison between A. niger and P. roqueforti on propionic acid-free bread showed, even after 5 days, 6 points covered by A. niger, and even 10 points by P. roqueforti. The inoculated bread slices, already after 2-4 days, had mycelia of P. roqueforti. In order to inhibit this mold fungus growth, a concentration of greater than 0.4% w/w propionic acid (based on the initial flour weight) was required.

When comparing the mycelial growth of A. niger on breads contained in the dough propionic acid and acetic acid formed according to the invention, after 8 or after 9 days of the challenge experiment it was found that the growth of A. niger could be suppressed even by concentrations of 0.3% propionic acid or a concentration of 0.2% w/w propionic acid and 0.2% w/w acetic acid (based on the initial flour weight) up to the end of the study.

Using the lactobacilli selected according to the invention, amounts of propionic acid of 0.1 to 6% in the sour dough were able to be achieved. However, as the challenge experiments showed, even at a content of 0.2% propionic acid and 0.2% acetic acid, the growth of the mold fungus A. niger was able to be completely suppressed. The co-fermentation according to the invention with lactobacilli which, inter alia, leads both to the formation of acetic acid and propionic acid, therefore shows a marked advantage and an improved preservation compared with a comparable chemical addition of at least 0.4% propionic acid in order to achieve comparable preservation results.

In summary, it was found that direct enrichment of acetic acid and propionic acid in the sour dough via an anaerobic metabolic pathway using lactic acid bacteria is an effective method and a marked improvement in bread preservation can be achieved using it.

A further particularly noteworthy advantage is that differences between the chemically added propionic or acetic acid and the propionic or acetic acid formed by fermentation with respect to odor could be established to the benefit of the propionic acid formed by fermentation. Therefore, a higher acceptance by the consumer for the preservation by fermentation using the cultures according to the invention may be expected.

Further advantages which are offered by the co-fermentation according to the invention for forming a biological protection by, inter alia, acetic acid and propionic acid, are, firstly, the short fermentation time and, secondly, the possibility of product protection not needing declaration.

### EXAMPLES

### 1. Use of Some Selected Lactobacilli for Formation of Propionic Acid in Wheat Doughs

For these experiments, the following type strains were used Lb. buchneri DSMZ 20057, Lb. parabuchneri DSMZ5707 and Lb. diolivorans DSMZ 14421. They were morphologically characterized in order to be able to enumerate them in a manner differentiated on the basis of colony shapes.

For adaptation of the lactobacilli to the dough medium, firstly in each case 1% overnight culture of Lb. buchneri, Lb. buchneri, Lb. parabuchneri and Lb. diolivorans were added to the dough and incubated at 28° C. for 24 h. Using this starter dough, sterilized wheat sour doughs were inoculated in such a manner that, based on the total amount, 10% dough containing Lb. diolivorans was used in each case in combination with 10% dough containing Lb. buchneri/Lb. parabuchneri. The doughs were stored anaerobically in gas-tight bags at 28° C. and the amount of organic acids and also the pH and acidity were determined.

In table 1, the results of the fermentations are shown. In the experimental batches described, up to 0.2% propionic acid was formed in the dough. After 14 days, similar amounts of propionic acid and acetic acid were found in all doughs. In the wheat sour dough containing Lb. parabuchneri and Lb. diolivorans, after 7 days still no propionic acid was recorded. The decrease in the amount of lactic acid was detectable in all experimental batches. The pH was, after 14 days, in all fermentations, 3.47, and the Sr° between 41 and 42 (0.1 n NaOH/10 g). The viable cell counts after 7 days were 10⁷ CFU/g.

TABLE 1
Overview of the organic acids during propionic acid
fermentations by Lb. buchneri/Lb. parabuchneri
in combination with Lb. diolivorans

| Time t | Lb. buchneri and | | Lb. parabuchneri and | |
|---|---|---|---|---|
| [d] | Lb. diolivorans | | Lb. diolivorans | |
| 0 | - | propionic acid | - | propionic acid |
| | 2 g of | acetic acid | 2 g of | acetic acid |
| | 34 g of | lactic acid | 34 g of | lactic acid |
| 7 | 1.6 g of | propionic acid | - | propionic acid |
| | 2.2 g of | acetic acid | 2.1 g of | acetic acid |
| | 30.44 g of | lactic acid | 29.24 g of | lactic acid |
| 14 | 2.0 g of | propionic acid | 2.1 g of | propionic acid |
| | 2.0 g of | acetic acid | 2.0 g | acetic acid |
| | 29.73 g of | lactic acid | 28.64 g of | lactic acid |

### 2. Formation of Acetic Acid and Propionic Acid in Rye Doughs

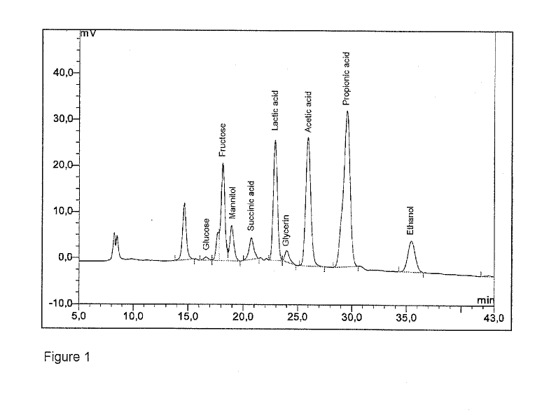

In a further exemplary embodiment, L. buchneri and L. diolivorans were inoculated in a sour dough consisting of whole grain rye flour and water and incubated anaerobically at room temperature. After three months, the sour dough was exposed to air and, within the next 4 weeks, displayed no sign of growth of molds or yeasts. After the three months, likewise, by means of HPLC (separation at 70° C. using Merck RT300-7.8 OAKC-column at a flow rate of 0.4 ml per min and 0.01 N sulfuric acid as eluent and RI detection) the concentration of acetic acid was determined at 15 g/kg and propionic acid at 19 g/kg. The chromatogram of this separation is enclosed as FIG. 1.

1. A sour dough starter for the production of sour dough comprising Lactobacillus diolivorans or derivatives thereof or recombinant lactobacilli, which are able to form propionic acid.

2. The sour dough starter for the production of sour dough as claimed in claim 1 comprising Lb. buchneri or Lb. parabuchneri.

3. A sour dough starter for the production of sour dough, characterized in that at least one first strain is selected from the group comprising Lactobacillus diolivorans and also derivatives thereof or recombinant lactobacilli, which are able to form propionic acid and wherein at least one second strain is selected from the group of the lactobacilli comprising Lb. buchneri, Lb. parabuchneri, Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confusa, P. acidilactici, P. parvulus and P. pentosaceus.

4. The sour dough starter for the production of sour dough as claimed in claim 1, characterized in that pure cultures selected from the group of the microorganisms are used.

5. A method for the production of sour dough by co-fermentation of lactic acid bacteria which comprises
selecting a first strain from the group comprising Lactobacillus diolivorans and also derivatives thereof or recombinant lactobacilli, which are able to form propionic acid, and
selecting at least one second strain from the group of the lactobacilli comprising Lb. buchneri, Lb. parabuchneri, Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confusa, P. acidilactici, P. parvulus and P. pentosaceus, wherein the first strain and the second strain of the lactic acid bacteria are mixed with sour dough components comprising at least flour and water, thereby producing the sour dough by co-fermentation.

6. The method for the production of sour dough as claimed in claim 5, wherein, after a 10 day fermentation, at least 0.3% propionic acid per liter of sour dough is detectable.

7. The method for the production of sour dough as claimed in claim 5, wherein, after a 10 day fermentation, at least 0.7% acetic acid per liter of sour dough is detectable.

8. A sour dough or sour dough concentrate produced using co-fermenting lactic acid bacteria, wherein at least one first strain is selected from the group comprising Lactobacillus diolivorans and also derivatives thereof or recombinant lactobacilli, which are able to form propionic acid, and wherein at least one second strain is selected from the group of the lactobacilli comprising Lb. buchneri, Lb. parabuchneri, Lb. plantarum, Lb. pontis, Lb. sanfranciscensis, Lb. crispatus, Lb. suntoryeus, Le. argentinum, Lb. helveticus, Lb. paralimentarius, Lb. fermentum, Lb. paracasei, Lb. frumenti, Lb. alimentarius, W. cibaria, W. confusa, P. acidilactici, P. parvulus and P. pentosaceus.

9. A sour dough or sour dough concentrate comprising Lactobacillus diolivorans and at least one strain selected from Lb. buchneri or Lb. parabuchneri.

10. A sour dough concentrate produced by concentrating from the sour dough as claimed in claim 8, characterized in that, per liter of concentrate, at least 0.7% of acetic acid and/or at least 0.3% of propionic acid are detectable.

11. The use of sour dough starter for the biological preservation of bread or baked goods comprising mixing Lactobacillus diolivorans or derivatives thereof or recombinant lactobacilli with sour dough components comprising at least flour and water, and observing a decrease in growth of molds and yeasts during shelf-life of the bread or baked goods, thereby using the sour dough starter for the preservation of resulting bread or baked goods.

12. The use of lactobacilli strains for the production of dough, sour dough, sour dough starter cultures and/or baked goods, characterized in that the lactobacilli strains are able to form 1,2-propanediol, propanediol, acetic acid and propionic acid from lactic acid.

13. The use of lactobacilli strains as claimed in claim 12, characterized in that the lactobacilli strains are selected from the group consisting of Lactobacillus diolivorans and also Lactobacillus diolivorans derived therefrom or recombinant Lactobacillus diolivorans.

14. Sour dough starter for production of sour dough as claimed in claim 2, wherein pure cultures selected from the group of microorganisms are used.

15. Sour dough starter for production of sour dough as claimed in claim 3, wherein pure cultures selected from the group of microorganisms are used.

16. Sour dough or sour dough concentrate as claimed in claim 9, wherein at least 0.7% of acetic acid or at least 0.3% of propionic acid is detectable per liter of concentrate.

17. The use of sour dough starter for the biological preservation of bread or baked goods as claimed in claim 11, comprising mixing Lb. buchneri or Lb. parabuchneri with the components.

18. Use of sour dough starter for biological preservation of bread or baked goods wherein at least one first strain is selected from the group comprising Lactobacillus diolivorans and also derivatives thereof or recombinant lactobacilli, which are able to form propionic acid and wherein at least one second strain is selected from the group of the lactobacilli comprising Lb. buchneri, Lb. parabuchneri, Lb. plantarum, Lb. pontis, Lb. sanfranciscensis, Lb. crispatus, Lb. suntoryeus, Leuconostoc argentinum, Lb. helveticus, Lb. paralimentarius, Lb. fermentum, Lb. paracasei, Lb. frumenti, Lb. alimentarius, Weissella. cibaria, W. confusa, Pediococcus. acidilactici, P. parvulus and P. pentosaceus.

19. The use of the sour dough or the sour dough concentrate as claimed in claim 8, for biological preservation of bread or baked goods.

20. The use of the sour dough or the sour dough concentrate as claimed in claim 9, for biological preservation of bread or baked goods.
